(19) United States
(12) Patent Application Publication
(10) Pub. No.: US 2009/0274769 A1
(43) Pub. Date: Nov. 5, 2009

(54) HEALING COMPOSITION

(76) Inventor: Alexandra Fregonese, Moncaut (FR)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/296,692

(22) PCT Filed: Apr. 10, 2007
(86) PCT No.: PCT/FR2007/000602

§ 371 Date: Oct. 10, 2008

(30) Foreign Application Priority Data
Apr. 10, 2006 (FR) 0603188

(51) Int. Cl. A61K 35/64; A61K 31/70; A61P 17/02

(52) U.S. Cl. 424/537; 514/23

(57) ABSTRACT

The invention relates to a ternary composition characterized in that it consists of a source of carbohydrates, a source of divided minerals, chosen from clays, and a source of fatty acids. It also relates to a healing composition comprising said ternary composition. In said compositions, the source of carbohydrates is chosen from honeys and the source of fatty acids is chosen from oils rich in linoleic acid and linolenic acid.

The present invention relates to the field of healing of dry and/or wet epithelial tissues, and more particularly to wound healing compositions and to dressings which can be used on wounds and regions of lesioned skin and/or mucous membranes, such as burns, decubitus ulcers and necrosed and deep wounds.

Numerous dressings currently exist on the market or are being tested, in particular dressings based on hydrocolloids, hydrogels, calcium alginates, charcoal dressings, dressings based on polysaccharides, hydrofiber dressings, hydrocellular dressings, paraffin gauze dressings or semipermeable films.

Despite major research studies, and in particular the development of hydrocolloid dressings, wound healing remains a major problem and more particularly the healing of decubitus ulcers and of deep, necrosed and exudative wounds.

Indeed, the various phases of the progression of the condition of a wound, from the inflammatory phase to the proliferation phase, via the detersive phase, require properties which are sometimes contradictory, which range from absorbent properties to detersive properties.

Furthermore, for all the phases to occur in a satisfactory manner, it is necessary for the wound covering to be permeable to oxygen and to water vapor, but preferably impermeable to bacteria, in order to avoid any contamination of the wound by exogenous bacteria.

A natural composition for cosmetic treatment is known from RO 119065. A composition in gum form containing, inter alia, a plant extract, bee honey and clay is described therein. It is stated that this product may have a wound healing effect. A gum is characterized by the presence of abrasive particles, consisting here of plant extracts incorporated in powdered form, which have the role of generating abrasion in order to remove the dead cells from the epidermis. This document does not disclose or suggest a smooth composition, without abrasive particles and gentle on lesioned skins, comprising clay, honey and vegetable oil, or the use of such a composition for the healing of lesions which may involve a loss of material, said wound healing using a process very different from that used during traditional wound healing.

The invention allows a satisfactory response to all the needs, regardless of the stage of progression of the wound and significant promotion of wound healing.

The invention consists of a ternary composition characterized in that it consists of a source of carbohydrates, a source of divided minerals and a source of fatty acids and in that it does not contain abrasive particles.

The expression without abrasive particles is understood to mean a composition containing no plant or mineral in powdered form.

The expression source is understood to mean a complex substance which, by analogy with the use of the term source in medicine, for example in the expression source of nutritive elements, will make it possible to provide certain elements which will diffuse from said source.

The source of carbohydrates is chosen from honeys.

The source of divided minerals is chosen from clays.

The source of fatty acids is chosen from oils rich in linoleic and linolenic acids.

The source of fatty acids is chosen from camelina oil, musk rose oil, evening primrose oil, safflower oil, blackcurrant seed oil, sunflower oil, wheat germ oil, borage oil and fish oils.

The invention relates to a ternary clay, honey and oil composition containing no abrasive particles.

The ternary composition according to the invention has wound healing properties for all epithelial tissues whether they are wet or otherwise (skin, buccal mucosa, vaginal and/or urinary mucosa) and regardless of the origin of the lesion, a traumatic and/or pathological origin for example due to an eczema, a psoriasis or an impetigo.

The invention also relates to the use of said composition for the preparation of a wound healing composition.

It also relates to a wound healing composition comprising, as active ingredient, a ternary composition as defined above.

The expression wound healing composition is understood to mean any composition intended to be applied to a wet or nonwet epithelial tissue, a mucous membrane or a skin, in an appropriate galenic form, namely a spray, a patch, a cream, a liquid, a paste or a microencapsulation.

The source of carbohydrates may be honey.

The chemical composition of honey varies according to its floral or geographic origin. Honey predominantly consists of carbohydrates 75%-80% (mainly glucose and levulose), essential amino acids, trace elements, mineral salts: calcium, chlorine, magnesium, potassium, iron, manganese, copper, silicon, sulfur, vitamins B1, B2, B3, B5, B6, B8 and PP, “antibiotic” factors and water.

Other substances, such as proteins and enzymes are also present.

The higher the fructose content of honey, the less it crystallizes; it is honey which is liquid, clear and fluid (example: acacia honey).

The higher its glucose content, the more it crystallizes and the thicker its consistency.

Rich in carbohydrates (¾ of its weight), low in sucrose, its assimilation produces no toxin in the body. Water is present in a nonnegligible quantity since its average content (it can vary) is 17.2% (no more than 21%).

Carbohydrates constitute the greatest part of honey. Monosaccharides (glucose and levulose) are present which represent 85% to 95% of the honey sugars, but it is levulose which is almost always predominant, with a content of 38% of the weight of honey, while the glucose content is 31%.

Sucrose (1.5%) and maltose (7.5%) are also present therein as well as other sugars present in traces: isomaltose, nigerose, turanose, maltulose, isomaltulose, leucrose, kojibiose, neotrehalose, gentiobiose, laminaribiose, melezitose, eriose, 1-kertose, dextrantriose, raffinose, isopanose, isomaltotetraose, 6-a-glucosylsaccharose, arabo-galactomannan, maltotriose, isomaltopentaose, panose, isomaltotriose, 3-a-isomaltosylglucose, centose.

The presence of levulose and glucose is mainly due to the action of invertase on sucrose.

Indeed, sucrose is dextrorotatory. When it is hydrolyzed, a mixture of equimolar quantities of D(+)-GLUCOSE and D(−)-FRUCTOSE is obtained: the levorotation of the fructose is therefore greater than the dextro-rotation of the glucose, such that the mixture obtained is levorotatory, which earned the name of invert sugar. SUCROSE+WATER=GLUCOSE+FRUCTOSE.

Honey also contains acids.

The most predominant is gluconic acid whose origin is thought to be a bacterium, called gluconobacter, which, during the maturation of honey, is thought to convert glucose to gluconic acid.

About twenty organic acids are also present therein, such as acetic acid, citric acid, lactic acid, malic acid, oxalic acid, butyric acid, pyroglutamic acid and succinic acid.

Traces of formic acid (one of the constituents of venom), hydrochloric acid and phosphoric acid are present therein.

Other compounds, the lactones, whose presence is constant, also have an acid functional group. The pH, which may vary from 3.2 to 4.5, is on average equal to 3.9.

The content of inorganic matter or ash is less than 1% (it is in general of the order of 0.1%).

Potassium, calcium, sodium, magnesium, copper, manganese, chlorine, phosphorus, sulfur and silicon as well as more than thirty trace elements are present therein in order of importance.

Their content depends on the plants visited by the bees and the type of soil in which they grow.

Proteins are present in a small quantity (1.7 grams per kilogram of honey, that is a content of 0.26%) and the nitrogen content is negligible (of the order of 0.041%).

This includes essentially peptones, albumins, globulins and nucleoproteins which are obtained either from the plant or from the bee.

There are also free amino acids including proline which are derived from the salivary secretions of the bee.

Numerous enzymes exist in honey: invertase, α-amylase, α-glucosidase and glucose oxidase capable of converting glucose to gluconic acid.

Honey also contains a catalase and a phosphatase.

It additionally contains very few vitamins, mainly the B (B1, B2, B3, B5, B6, B8, B9) vitamins provided by pollen.

It also comprises traces of lipids probably supplied by the wax which escapes filtration and aromatic substances.

The weight is assessed with a densimeter and an average of 1.4225 at 20° C. can be accepted.

While all honeys can be used, lavender honey will be preferably used.

The expression divided minerals is understood to mean minerals whose size is of the order of the micron, chosen from metal oxides or hydroxides, preferably alkaline-earth metals such as Mg and Ca, but also carbonates, phosphates and silicates.

The source of divided minerals may thus consist of clays or argillaceous rocks.

Several forms of clay exist: illite, chlorite, glauconite, kaolinite, montmorillonite, attapulgite and sepiolite.

The argillaceous state is characterized by the presence of extremely divided minerals, having a flattened shape and thereby having plastic and absorbent properties.

These minerals are hydrated silicates having the phyllitic or fibrous texture, which are sufficiently stable in the natural conditions to preserve their state of division without changing notably.

Argillaceous rocks are mixtures of minerals in which the major fraction has a size corresponding to the argillaceous state, that is substantially 2 microns, and containing, in addition to the specific minerals which confer their character on them, various minerals which may have any of the properties of the argillaceous state.

Among the most common nonspecific minerals are the oxides or hydroxides of a number of elements, silica, feldspars, carbonates and phosphates.

Clays also contain a moderately large quantity of organic matter, in particular in deposits located close to the surface of the ground.

Clays are therefore mixtures of minerals which can be schematically divided into two groups:
argillaceous minerals which confer on clay its plasticity (mainly silicates in the form of sheets)
accessory minerals, in particular iron, aluminum and magnesium oxides and hydroxides.

Clays, at the chemical level, are hydrated aluminosilicates into which foreign mineral elements which give various colorations of clay are embedded.

The size of the various mineral elements is about 2 microns. These hydrated silicates have a phyllitic texture (in sheet form) or a fibrous texture and thus confer a moderately high plasticity on the clay.

It exists in 2 or 3 layers. Clays are classified by family according to their mineral elements and their crystalline structure.

Clay is a soft rock that is rich in minerals and trace elements. Its color varies according to the iron oxides which it contains.

It has a high absorbent power.

While all clays can be used, montmorillonite will be preferably used because of its natural richness in magnesium (10%).

The source of fatty acids is chosen from oils rich in linoleic and linolenic acids.

The fatty acids are derived from the degradation of lipids during digestion by enzymes to make them assimilable by the blood and lymphatic systems. These fatty acids exist in the form of organic molecules which may be classified into three families, butyric acid (saturated), oleic acid (monounsaturated) and linoleic acid (polyunsaturated). Some of these fatty acids are said to be essential because they cannot be produced by the body or are produced in a very small quantity.

Among these, two polyunsaturated fatty acids, linoleic acid and linolenic acid, are responsible for two noncommunicating metabolic chains, omega-3 and omega-6.

These two fatty acids are involved in the process for the production of prostaglandins and thereby in the reproductive and growth processes, formation of cells, integrity of the skin, renal functions, inflammatory reactions, allergic reactions, immune reactions and platelet aggregation.

The oils are chosen from oils rich in linoleic and linolenic acids, such as some vegetable oils, camelina oil, musk rose oil, evening primrose oil, safflower oil, blackcurrant seed oil, sunflower oil, wheat germ oil, shea oil or borage oil, and fish oils.

Oils that are liquid at 20° C. will be preferred, but depending on the desired final texture, it will be possible to use solid oils such as palm oil and shea butter for example.

Most vegetable oils rich in linoleic and linolenic acids have compositions which may be generalized as follows:

Musk rose oil contains roughly 1% of unsaponifiable matter: linoleic acid (40%), linolenic acid (40%), oleic acid (15%), palmitic acid (3%).

Its principal components are:
Fatty acid triglycerides (mainly polyunsaturated)
Proportion of fatty acids:
Saturated fatty acids
Palmitic acid (C16:0): 3.4 to 8%
Stearic acid (C18:0): 1.6 to 3.0%
Arachidic acid (C20:0): 0.2 to 1.0%
Monounsaturated fatty acids
Oleic acid (C18:1): 13 to 18%
Palmitoleic acid (C16:1): traces
Polyunsaturated acids
Linoleic acid (C18:2): 41 to 51%
Linolenic acid (C18:3): 24 to 39%

Other components:
Unsaponifiable matter: 0.8 to 1.6%

In one embodiment, the ternary composition is characterized in that it comprises from 4 to 40% of clay, from 8 to 35% of honey and from 8 to 40% of oil by mass relative to the total weight of the composition.

It will be possible for the ternary composition according to the invention to be used as active ingredient for the preparation of a wound healing composition.

The invention therefore relates to a wound healing composition containing a ternary composition as defined above.

It also relates to a wound healing composition, characterized in that it additionally contains exogenous water.

It also relates to a wound healing composition, characterized in that it additionally contains metal oxides.

It also relates to a wound healing compositions characterized in that it additionally contains emulsifiers.

It also relates to a wound healing composition, characterized in that it additionally contains wax.

It also relates to a wound healing composition, characterized in that it additionally contains antioxidants.

It also relates to a wound healing composition, characterized in that it additionally contains preservatives.

The metal oxides will be chosen from the group consisting of zinc, titanium and cerium oxides.

The emulsifier is chosen from the group consisting of sorbitan oleate, polysorbates, cyclopentasiloxane, polyethylene glycols (PEG), polypropylene glycols (PPG), copolyol dimethicone, lauryl methicone copolyol or, without thereby being exhaustive, alkyl methicones.

The antioxidant is chosen from the group consisting of butylhydroxytoluene, sodium metabisulfite or sodium sulfite.

The preservative is chosen from the group consisting of phenoxyethanol, parabens, sequestrants such as EDTA, sorbic acid, benzoic acid, sodium lauroyl lactylate, glyceryl caprylate, pentylene glycol or natural preservatives based on essential oils.

Wax is a thermoplastic material, that is synthetic (cetareth alkyl methicone, PEG stearate, cetyl alcohol, stearyl alcohol, cetearyl alcohol, PEG, stearate sodium, polyoxyethylene, sodium stearate), that is a mixture of substances of mineral origin (paraffin, ceresin), that is of plant origin (carnauba wax, hydrogenated coconut oil) and that is of animal origin (beeswax) which contains propolis, pollen grains and chrysin and between 90 and 95% of pure wax. While the composition varies according to its region of origin, it has however a chemical composition which corresponds to 12 to 12.5% of hydrocarbon, from 13 to 13.5% of free fatty acids, 72% of esters of aliphatic alcohols, 0.8% of cholesterol ester, 0.6% of lactone and 2% of water.

The total quantity of water present in a formula will depend on the galenic form chosen, the destination site and the pathology or the lesion to be healed. In any case, it will be possible for it to comprise between 0 and 95% by mass relative to the total weight of the composition.

The total quantity of metal oxide present in the formula of the composition is between 3 and 10% by mass relative to the total weight of the composition.

The total quantity of emulsifier present in the formula will, for its part, depend on the galenic form chosen, the destination site and the pathology or the lesion to be healed and will comprise between 5 and 50% by mass relative to the total weight of the composition.

The total quantity of preservative present in the formula is between 0.05 and 2% by mass relative to the total weight of the composition.

The total quantity of wax present in the formula is between 0 and 30% by mass relative to the total weight of the composition.

The wound healing composition according to the invention is prepared so as to be packaged in the form of a paste, a patch, a liquid, agar, a moderately thick emulsion or a microencapsulation so as to be attached to a support of the dressing type.

It is used to cover lesioned regions of the skin and/or the mucous membranes, and in this regard, the invention also consists of a dressing, characterized in that it comprises the ternary composition according to the invention.

The invention also relates to a dressing comprising a ternary composition comprising clay, honey and oil.

It relates to a dressing comprising a ternary composition consisting of 4 to 40% of clay, 8 to 35% of honey and 8 to 40% of oil.

The invention also relates to a cosmetic composition, characterized in that it comprises a ternary composition according to the invention.

It also relates to a medicament comprising a bioresorbable ternary composition consisting of a source of carbohydrates, a source of divided minerals chosen from clays and a source of fatty acids, for the wound healing treatment of a cutaneous lesion which is capable of exhibiting a loss of material.

The present invention also relates to a medicament comprising a bioresorbable ternary composition consisting of a source of carbohydrates, a source of divided minerals chosen from clays and a source of fatty acids, for the wound healing filling of a cutaneous lesion exhibiting a loss of dermal material by centrifugal neo-dermogenesis, that is to say from the base of the wound to the top or the surface thereof.

It also relates to a medicament comprising a bioresorbable ternary composition consisting of a source of carbohydrates, a source of divided minerals chosen from clays and a source of fatty acids, for the wound healing filling of a cutaneous lesion exhibiting a loss of dermal material by a pharmacological mode of action other than surface cyto-stimulation or the activation of enzymes such as inhibition or induction of the collagenolytic or elastase activities.

It finally relates to a dressing, characterized in that it comprises a medicament according to the invention.

The composition according to the invention is prepared according to a process which comprises the steps of mixing and dissolving in the hot state with vigorous stirring with the aid of an emulsifier located at the bottom of the tank and oversized in relation to the capacity of the mixer.

The mixer should additionally be equipped with scraping blades because of the viscosity of the product and a possibility of mixing under vacuum.

It will be advisable to add the fatty acid fraction to the clay in order to trap it in the clay, and then to bind the whole with honey after having ensured that the preparation is homogeneous.

When the ternary composition is used as active ingredient in a wound healing composition, the other ingredients are then successively added to the ternary composition.

The compositions according to the invention, ternary composition and/or wound healing composition, have viscosities of between 390 000 cps needle 7, speed 6 at 20° C. and 1300 cps needle 3, speed 60 at 21° C.

The physiological wound healing process, when there is a wound or an ulcer, with loss of material (dermal loss), is customarily described as a centripetal process and is performed in several phases from the edges of the wound to its center.

With a pharmacological agent such as an activator of cell anabolism (activation of the synthesis of collagens and of other proteoglycans, and/or of the synthesis of glucosaminoglycans of the extracellular matrix of the dermis), this centripetal wound healing process is often increased.

This physiological wound healing process is very often associated with a loss of volume. This loss of volume results:
from the sagging of the edges of the wound given the time required for the wound healing,
from the rate of re-epithelization which often overtakes neo-dermogenesis (closure of the wound before complete recovery of the lost volume), especially if there is anabolic pharmacological stimulation (cyto-stimulation),
but also from the physiological process of centripetal dermal neosynthesis (production of the dermis from the edges for the base of the fleshy bud).

The composition according to the invention allows the wound healing to take place, not in a centripetal manner, but by centrifugal dermogenesis, that is to say from the base of the wound to the top of the wound, without loss of volume, and with a difference in quality and in maturation of the neo-dermis from the base to the top of the wound. This mode of wound healing is in correlation with the physical variation of the composition during wound healing and runs counter to the normal or accelerated physiological process by a pharmacological agent.

No loss of volume, in relation to a sagging of the edges of the wound, was observed in the regions treated with the composition according to the invention.

Furthermore, analysis over time of the variation of the surface of the lesion, treated with a composition according to the invention, showed that no surface cyto-stimulant effect, relating particularly to the epidermis (which would have generated a re-epithelization and therefore a reduction in the top of the lesion) was observed. This analysis also showed that no inhibiting or inducing effect of the physiological collagenolytic or elastase activities was observed.

The composition according to the invention was developed with the aim of providing a novel and different response to the management of a wound.

The structure of said composition allows the maintaining, by filling, of a traumatic cutaneous depression such as an ulceration or a wound with a loss of material (with dermal loss), and then the attachment and sequestering, like a biological sponge, of the cicatricial exudates produced by the ulcerated or injured skin, during the wound healing process, and the release of said products in a third stage.

Upon application, its structure and its pasty texture will allow the filling of the cutaneous lesion and the effective maintaining of the distance between the edges of the wound.

Its absorption and adsorption properties confer an evolutive plasticity on it. Over time, the composition according to the invention will sag and become concentrated at the bottom of the wound, simultaneously concentrating the physiological entities contained in the previously sequestered exudates. Its slow and uniform resorption will make it possible to gradually release these physiological compounds on the base of the wound without disrupting the formation of the neodermis.

The composition according to the invention or the dressing according to the invention is slowly and uniformly resorbed and allows the maintaining or the renewal of the tissues.

The gradual release of nutritive elements supports and accentuates all the wound healing stages by intervening in the process early.

The source of carbohydrates or the honey is involved in all the stages of the wound healing in the inflammatory phase, in the detersive phase and in the proliferation phase.

It allows control of microbial proliferation by equilibrating the cutaneous ecosystem, it activates the inflammatory phase by virtue of the polysaccharides and feeds the cells by virtue of the sugar which it contains.

The clay, by virtue of its very high adsorbent power, has a capacity to bind and therefore to neutralize toxins and bacteria, and in particular the toxic derivatives from putrefaction (the purines derived from the hydrolyzing degradation of the nucleoalbumins of the body), the bacterial toxic residues during infection and the toxic products brought by living bacteria.

The composition according to the invention or the dressing according to the invention acts at the three stages of wound healing which are inflammation, detersion and proliferation.

During hemostasis, the composition or the dressing according to the invention has a filling role.

On contact with the serous fluids, it forms a stable mechanical matrix which offers the pressure required for vasoconstriction and clotting.

There is aggregation of the platelets and the formation of fibrin (this whitish and elastic insoluble filamentous globulin which forms a network and whose nodes consist of platelet aggregates).

The wound healing composition or the dressing pulls the lips apart in order to promote simultaneous tissue reconstruction from the bottom and the edges simultaneously.

The presence of polysaccharides contained in honey then intensifies the inflammatory reaction.

The polysaccharides have the property of stimulating the defense system, the immune system by activation of the macrophages (the Langherans' cells for the skin).

The membrane of the Langherans' cells carries, at its surface, a receiving site into which a polysaccharide molecule perfectly fits.

This results in the activation of the macrophage and consequently the initiation of the immune cascade.

This leads to a high inflammatory reaction. This inflammatory response is based on 4 elements (the Celsus quadrilateral) which are: pain, heat, redness and swelling with:
vasomotor phenomena (redness and edemas)
cellular phenomena (diapedesis, that is to say migration of white blood cells across the wall of the vessels). The lymphocytes, agents of immunity, and the monocytes, precursors of the macrophages, thus migrate and bind to the site to be treated,
tissue phenomena,
immunological phenomena.

It is the quality of this stage which determines the normal course of the other wound healing phases.

This phase generates a good blood supply which will shorten the detersion time and accelerate healing by leaving a better scar.

In the detersion phase, the increase in capillary permeability promotes the passage of blood plasma with antibodies, leucocytes and macrophages to the traumatized region.

Thus, the necrosed tissues, the foreign bodies and the microbes are removed and destroyed by phaghocytosis and proteolysis.

The shortening of the detersion phase is vital because it accelerates the healing and gives better wound healing.

It is therefore essential to offer at this stage, and with the aim of establishing the benefits of a quality inflammatory phase, an effective action at the time of detersion.

The efficacy of the wound healing composition or the dressing depends on the clay at this stage. If the clay is insoluble in water, it has on the whole a very high absorbent power.

Also, on contact with the exudates, it absorbs them, purifying the medium and limiting bacterial proliferation.

Moreover, it maintains a wet medium which promotes wound healing and autolytic detersion.

The composition or the dressing according to the invention has high detersive properties and offers the great advantage of being permeable to oxygen and to water vapor but not to bacteria, which distinguishes it in particular from hydrogels whose major disadvantage is the nauseating odor characteristic of microbial development.

This barrier function is due to the simultaneous presence of clay, honey and fatty acids.

By virtue of its adsorbent power, the power of clay is unquestionable against viruses and bacteria.

The microorganisms are trapped and the enzymes and toxins suffer the same fate.

Once immobilized, they lose their action and are evacuated.

This synergy of active agents offers major advantages at the detersion stage which are on top of the detersion powers constituted by the maintaining of a wet medium by controlling the exudate.

Clay absorbs eight times its weight of water.

It possesses the highest CEC (Chemical Exchange Capacity) of all the argillaceous minerals.

Accordingly, the composition or dressing according to the invention will vary with the absorption of serous fluids.

This plasticity is linked to the crystalline structure of argillaceous minerals and especially of silicates.

The first absorptions will maintain the moisture in contact with the walls while preserving a considerable pressure because of the fact that only the peripheral and external parts of the wound healing composition or the dressing will be impregnated.

Next, as the absorption progresses, the wound healing composition or the dressing will become increasingly malleable, gradually reducing the pressure exerted on the walls in order to promote blood flow and enter into the proliferation phase.

During the proliferative phase the body begins to make up for the loss of substance with a new tissue.

For this purpose, the fibroblasts first of all produce mucopolysaccharides which will serve as matrix for the production of the collagen fibers of the connective tissue.

The mineral elements of clay absorb the photon energy.

After storage, this energy is retransmitted to their surface, forming primary protoorganisms.

It is on the coefficient of ion exchange (specific to each clay) by supply of mineral elements loaded with energy that the efficacy depends.

Clays are electrically charged bodies. They possess, at the surface, ions (negative charges) that are neutralized by the presence of opposite positive ions.

The substitution causes the replacement of metal ions by cations of a lower charge.

At this stage, the composition or the dressing according to the invention is filled with exudates and with serous fluid.

However, rocks such as clay fragment under the action of water molecules.

There is a departure of silica ion and a binding of water which modifies the equilibrium of the rock.

Water causes hydrolysis, the siliceous framework of the clay becomes cavernous and breaks.

Hydrolysates such as aluminum hydroxide are formed.

The ion-exchange reaction continues and gradually destroys the structure of the mineral.

The clay in the formulation or the dressing according to the invention is an open structure in the form of sheets which form a complex with the organic compounds of honey.

When the structure is destroyed, the organic compounds of the honey are released.

This physical change thus produces the phenomenon of bioresorbability of the matrix and a range of chemical phenomena which contribute to nutrition.
release of sugars.

It causes a sudden increase in the level of glutathione which exercises the role of hydrogen transporter in the body.

This plays an important role in the oxidoreduction process in the body, stimulating the division and growth of the cells.

Vitamin P promotes vascular permeability.

The calcium, phosphorus and iron contained in both honey and clay then act on blood fluidity by increasing the hemoglobin level (precious factor which adds to the action of B12—essential as regards cell division, the synthesis of nucleic acids and numerous enzymatic processes—and folic acid—Vitamin B9 (spinach leaf extract), it is recognized as a factor that is necessary for normal growth and hematopoiesis (formation of blood cells)—which results in better transport of oxygen to the cells.

The epithelial tissues and the subcutaneous nerve branches are fed, the damaged cells are regenerated and the wound healing is promoted.

At this stage, the polyunsaturated fatty acids also become involved.

The unsaturated fatty acids enter into the composition of the membrane of nerve cells in a proportion of a third.

It is through these membranes that all messages pass from one cell to another.

When the proportion of fatty acid is insufficient, the membranes are more fragile and are gradually destroyed.

They are moreover involved in the constitution of the cell membranes, the enzymes and play a protective role for the vessels.

In the hemostasis and detersion phases, these active agents, in lipid form, are not released because of the fact that clay retains them between these sheets.

It is therefore necessary for it to have captured the exudates through its absorbent power, for the siliceous framework to be destroyed and for the release to occur on contact with water.

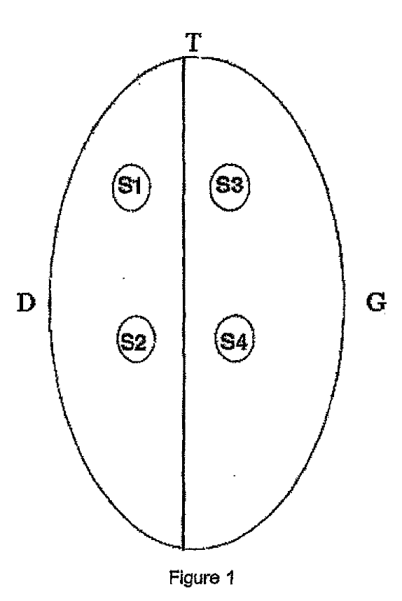

FIG. 1 represents the scheme for the sites of biopsies performed on each animal. The letter G indicates the left flank of the animal, the letter D its right flank and the letter T its head. The site S1 corresponds to the site treated with the placebo (petroleum jelly), the site S2 to the site treated with Bepanthen and the sites S3 and S4 correspond to the sites treated with the composition according to the invention.

### EXAMPLE 1

### Preparation and Use of the Wound Healing Composition According to the Invention

A ternary composition comprising 35% of clay, 30% of honey and 35% of oil according to the invention is obtained by mixing, after successive introductions, in the hot state, of various ingredients.

A composition having a viscosity of about 400 000 cps needle 7, speed 6 at 20° C. is obtained after mixing in a scraping blade mixer.

To obtain the wound healing composition:

In another tank, the dissolution of zinc oxide is prepared.

TANK 1: Water is placed in a tank at 55° C. and the zinc oxide is solubilized.

In TANK 2:—the ternary mixture is placed at 55° C. Into a CONTAINER at 55° C.: 13% of sorbitan oleate is placed, 4% of polysorbate 20 is added, BHT and the mixture of parabens and of phenoxyethanol chosen to preserve the mixture are introduced.

1—The contents of the container are integrated under vacuum into the tank 1 and left to rotate for 5 min at 4000 revolutions/min.

2—The contents of the tank 1 are then gradually added under vacuum, and with vigorous stirring, into the tank 2 and left to rotate for 20 min at 4000 revolutions/min.

A wound healing composition is obtained which has a viscosity of 1000 cps, needle 3, speed 60 at 21° C., which is then packaged in the form of patches, after coating and cutting.

### EXAMPLE 2

### Evaluation of the Mode of Action of the Wound Healing Composition According to the Invention

Pharmacology In Vivo: Cutaneous Ulcer Model

After a period of acclimatization of at least 5 days before the first day of the study, 4 cutaneous ulcers (Punch Biopsy 6 mm) were prepared as described below on 12-week old rats (male Sprague-Dawley rat, Rj: SD TOPS Han), according to Dorsett-Martin W. A. (2004).

One day before the experiment, the back of the animals was shaved with an electric razor, taking care not to damage the skin. On the first day of the experiment, under local anesthetic (lidocaine EMLA cream®) and under general anesthetic (Pentobarbital® 30 mg/kg i.p.), the skin, shaved beforehand, is cleaned with a liquid soap and then rinsed with purified water and dried using compresses impregnated with Betadine®.

After verifying the absence of response to interdigital pinching or to pinching in the region of the tail, 4 skin excisions (two per flank), schematically represented in FIG. 1, of 6 mm were made on each animal with the aid of a stylet for Punch biopsy (hole punch).

The punch biopsies were separated from the subcutaneous tissue with the aid of a sterile scalpel. The regions of the ulcers were cleaned with an antiseptic solution (Mercryl®), and then covered with a sterile compress held in place with aerated hypoallergenic sticking plasters.

From day 3 up to day 22, the ulcers were cleaned daily with physiological saline and treated once per day with the test element or the reference elements.

The test element (Product A) comprises a ternary composition according to claim 5 to which water, preservatives, titanium oxide, zinc oxide and flavorings have been added.

TABLE 1
Description of the test element and of the reference elements

| Products | Name | Supplier | Batch No. | Description |
|---|---|---|---|---|
| Test item | Product A | Laboratoire | 05G03 | Pasty form |
| | | Agelys | | |
| Negative | Medicinal | Laboratoire | 04050140/E | Cream |
| Reference | petroleum | Cooper | | |
| | jelly | | | |
| Positive | Bepanthen | Laboratoire | // | Cream |
| Reference | 5% ® | Roche-Nicolas | | |

### Histological Analyses

Histological sections were prepared using the microtome as 4-6 μm thick sections from paraffin blocks containing skin samples. These sections were stained with Masson's Trichrome (identification of the dermis and of the neodermis) according to the technique described below. After staining, microscopic analysis was performed by an experienced pathologist in the field either directly or in polarized light (mature collagen and immature neocollagen).

### Celestine Blue Solution:

25 g of ferric ammonium sulfate are dissolved in 500 ml of distilled water in the cold state. 2.5 g of Celestine blue are added, the mixture is brought to the boil and filtered. 70 ml of glycerine are added.

### Cole's Hematoxylin Solution:

15 g of Hematoxylin are dissolved in 250 ml of hot water. 50 ml of an iodine solution are added (1% in 95% Alcohol) and then 750 ml of a saturated potassium alum solution (K Al (S04)2: merck 1.01047.1000) are added. The mixture is rapidly filtered.

### Fushin Solution:

3 g of acid Fushin are mixed in the cold state with 597 ml of distilled water containing 3 ml of glacial acetic acid.

### Phosphomolybdate Solution:

6 g of phosphomolybdic acid are dissolved in 600 ml of water.

### Methyl Blue Solution:

12 g of Methyl blue are dissolved in 600 ml of distilled water containing 15 ml of glacial acetic acid.

### Method:

The 4-6 μm sections are fixed for 10 minutes in toluene and then for 1 minute in absolute alcohol and then twice 1 minute in distilled water. They are stained with the Celestine Blue solution for 5 minutes and then rinsed twice 1 minute with distilled water. They are stained with the Cole's hematoxylin solution for 5 minutes and then rinsed twice 1 minute in distilled water. They are stained with the Fushin solution for 5 minutes and then rinsed twice 1 minute with distilled water. They are stained with the Phosphomolybdate solution for 5 minutes, and then for 5 minutes with the Methyl blue solution. They are finally rinsed twice 1 minute with distilled water.

The coloration is fixed by a 2-minute treatment in a 1% acetic acid solution, and then by a bath in absolute alcohol for 1 minute and finally by a 1-minute treatment in toluene.

### Results:

The histological analysis was carried out as described in the method. The first staining (Masson's Trichrome) made it possible to assess the extent of the post-cicatricial neodermis, the loss of volume relating to this wound healing and the mechanical wound healing process.

It was observed that, typically, a scar treated with the placebo (petroleum jelly) generates a centripetal wound healing process (neodermogenesis) starting from the edges of the wound to the centre. This wound healing process induces a loss of volume by sagging of the edges of the wound.

It was observed that with a pharmacological agent of the cellular anabolism activator type (activation of the synthesis of collagens and other proteoglycans, or/and of the synthesis of glucosaminoglycans of the extracellular matrix of the dermis), this centripetal wound healing process is increased.

What characterizes a pharmacological mode of action is the preservation of the centripetal wound healing process from the edges of the lesion to its center. The trials showed that the wound healing process, increased by the pharmacological effect, occurred from the edges of the wound and that it caused a substantial loss of volume due to a sagging of the edges of the wound with tension of the skin.

This process was never observed during the histological analyses of the post-cicatricial regions treated with the product A.

No loss of volume, relating to a sagging of the edges of the wound, was observed on the same animals, in the regions treated with the product A. The wound healing process is clearly from the bottom to the top and not centripetal as in the previous cases. This wound healing mode is in correlation with the physical variation of the product A during wound healing and runs counter to the normal physiological process, or is accelerated by a pharmacological agent.

Taking these results into account, a second analysis in polarized light was carried out. The aim of this analysis was to check that the mode of wound healing observed indeed proceeded from the base to the top and not in a centripetal manner.

The confirmatory hypothesis was the following: If the wound healing process indeed proceeded from the bottom to the top, then the progression of wound healing implied that the base of the scar (deep dermis) had to have a much greater anteriority and therefore a much more marked organization than the top of the scar (superficial dermis and epidermis).

It is this parameter that was observed in polarized light. Mature collagen, properly organized as fiber in the extracellular matrix of the dermis, reflects polarized light while the neocollagen (procollagen) absorbs it due to a partial or incomplete organization.

The histological reference in this type of analysis is the organized mature collagen of the peripheral regions of the cicatricial region.

It was observed, on the samples, that polarized light is reflected by the base of the scar at the level of the deep dermis. This reflection is practically equivalent to that observed at the periphery and is only partial in the middle dermis.

The superficial dermis absorbs polarized light, reflecting a degree of organization that is far less advanced.

### CONCLUSION

The mode of healing, induced by the product A, of wounds with loss of material (dermal loss), indeed appears to be distinguishable from a mode of wound healing that is physiological or induced by a pharmacological process.

Unlike what is physiologically observed or after pharmacological cell stimulation, the analysis of the scars obtained after treatment with the product A shows an absence of loss of volume with an absence of sagging of the edges of the wound and in particular a very marked process of reorganization and maturation of the neodermis formed from the base of the cicatricial region to the top of this same region.

This maturation process “by stage” from the base to the top of the wound runs counter to the physiological centripetal wound healing process from the edges of the wound to its center observed with the placebo or the pharmacological agent.

The histological analysis of the cicatricial regions during this study made it possible to show that, in the same subjects, depending on whether the ulcerated region had been treated with the product A or with the pharmacological positive reference, the quality of the scars was very different.

On the basis of the observations of this study, it appears that the mode of action of the product A can be correlated with its physical variation in the wound, and that it is the main constraint modulating the wound healing process. This process runs counter to the physiological process or the process induced by a pharmacological agent, without thereby limiting the wound healing.

The other major point, which distinguishes the scars analyzed and obtained after treatment with the product A from the others, is the absence of loss of cutaneous volume associated with the wound healing process.

1. A ternary composition, characterized in that it consists of a source of carbohydrates, a source of divided minerals chosen from clays and a source of fatty acids and in that it does not contain abrasive particles.

2. The ternary composition as claimed in claim 1, characterized in that the source of carbohydrates is chosen from honeys.

3. The ternary composition as claimed in claim 1, characterized in that the source of fatty acids is chosen from oils rich in linoleic and linolenic acids.

4. The ternary composition as claimed in claim 1, characterized in that the oil is chosen from camelina oil, musk rose oil, evening primrose oil, safflower oil, blackcurrant seed oil, sunflower oil, wheat germ oil, borage oil and fish oils.

5. The ternary composition as claimed in claim 1, characterized in that it consists of 4 to 40% of clay, 8 to 35% of honey and 8 to 40% of oil by mass relative to the total weight of the composition.

6. A wound healing composition, characterized in that it comprises, as active ingredient, the ternary composition as defined in claim 1.

7. The wound healing composition as claimed in claim 6, characterized in that it additionally comprises exogenous water.

8. The wound healing composition as claimed in claim 6, characterized in that it additionally comprises a metal oxide chosen from zinc, cerium or titanium oxide.

9. The wound healing composition as claimed in claim 6, characterized in that it comprises an emulsifier chosen from sorbitan oleate, polysorbates, cyclopentasiloxane, PEGs, PPGs, copolyol dimethicone, lauryl methicone copolyol or alkyl methicones.

10. The wound healing composition as claimed in claim 6, characterized in that it comprises wax.

11. The wound healing composition as claimed in claim 6, characterized in that it comprises an antioxidant chosen from butylhydroxytoluene, sodium metabisulfite or sodium sulfite.

12. The wound healing composition as claimed in claim 6, characterized in that it comprises a preservative chosen from synthetic preservatives such as phenoxyethanol, parabens, sequestrants such as EDTA, sorbic acid, benzoic acid, sodium lauroyl lactylate, glyceryl caprylate, pentylene glycol or natural preservatives based on essential oils.

13. The wound healing composition as claimed in claim 6, characterized in that the total quantity of water present in the formula is between 0 and 95% by mass relative to the total weight of the composition.

14. The wound healing composition as claimed in claim 6, characterized in that the total quantity of metal oxide present in the formula is between 3 and 10% by mass relative to the total weight of the composition.

15. The wound healing composition as claimed in claim 6, characterized in that the total quantity of emulsifier present in the formula is between 5 and 50% by mass relative to the total weight of the composition.

16. The wound healing composition as claimed in claim 6, characterized in that the total quantity of preservative present in the formula is between 0.05 and 2% by mass relative to the total weight of the composition.

17. The wound healing composition as claimed in claim 6, characterized in that it contains from 0% to 30% of wax.

18. The wound healing composition as claimed in claim 6, characterized in that it comprises 35% of clay, 30% of honey and 35% of oil.

19. A process for preparing a ternary composition comprising the steps of mixing and dissolving in the hot state, with vigorous stirring, the fatty acid fraction to be entrapped in the clay, followed by an operation of binding the mixture obtained with honey.

20. A cosmetic composition, characterized in that it comprises a ternary composition as claimed in claim 1.

21. The use of a ternary mixture of clay, honey and oil for the preparation of a wound healing composition.

22. A dressing, characterized in that it comprises from 4 to 35% of clay, from 8 to 30% of honey and from 8 to 35% of oil.

23. The dressing as claimed in claim 21, characterized in that it comprises 8.5% of clay, 18% of honey and 23% of oil.

24. A medicament comprising a bioresorbable ternary composition consisting of a source of carbohydrates, a source of divided minerals chosen from clays and a source of fatty acids, for the wound healing treatment of a cutaneous lesion which is capable of exhibiting a loss of material.

25. A medicament comprising a bioresorbable ternary composition consisting of a source of carbohydrates, a source of divided minerals chosen from clays and a source of fatty acids, for the wound healing filling of a cutaneous lesion exhibiting a loss of dermal material by centrifugal neo-dermogenesis, that is to say from the base of the wound to the top or the surface thereof.

26. A medicament comprising a bioresorbable ternary composition consisting of a source of carbohydrates, a source of divided minerals chosen from clays and a source of fatty acids, for the wound healing filling of a cutaneous lesion exhibiting a loss of dermal material by a pharmacological mode of action other than surface cyto-stimulation or the activation of enzymes such as inhibition or induction of the collagenolytic or elastase activities.

27. A dressing, characterized in that it comprises the medicament as claimed in claim 24.
